(19) United States
(12) Patent Application Publication
(10) Pub. No.: US 2017/0119572 A1
(43) Pub. Date: May 4, 2017

(54) Urine Collection Assembly

(71) Applicant: George Mullings, Edmonton (CA)

(72) Inventor: George Mullings, Edmonton (CA)

(21) Appl. No.: 14/927,260

(22) Filed: Oct. 29, 2015

(51) Int. Cl. A61F 5/453; A61F 5/44

(52) U.S. Cl. CPC A61F 5/453; A61F 5/4408

(57) ABSTRACT

A urine collection assembly for collecting urine from a user includes a harness that may be worn. A receptacle is coupled to the harness and the receptacle may insertably receive a penis when the harness is worn. Thus, the receptacle may collect urine. A container is provided and the container may be worn. The container is in fluid communication with the receptacle and the container stores the urine.

### BACKGROUND OF THE DISCLOSURE

### Field of the Disclosure

The disclosure relates to collection devices and more particularly pertains to a new collection device for collecting urine from a user.

### SUMMARY OF THE DISCLOSURE

An embodiment of the disclosure meets the needs presented above by generally comprising a harness that may be worn. A receptacle is coupled to the harness and the receptacle may insertably receive a penis when the harness is worn. Thus, the receptacle may collect urine. A container is provided and the container may be worn. The container is in fluid communication with the receptacle and the container stores the urine.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a front perspective view of a urine collection assembly according to an embodiment of the disclosure.

FIG. 2 is a back view of an embodiment of the disclosure.

FIG. 3 is a front view of an embodiment of the disclosure.

FIG. 4 is a perspective in-use view of an embodiment of the disclosure.

FIG. 5 is a front perspective in-use view of an embodiment of the disclosure.

### DESCRIPTION OF THE PREFERRED EMBODIMENT

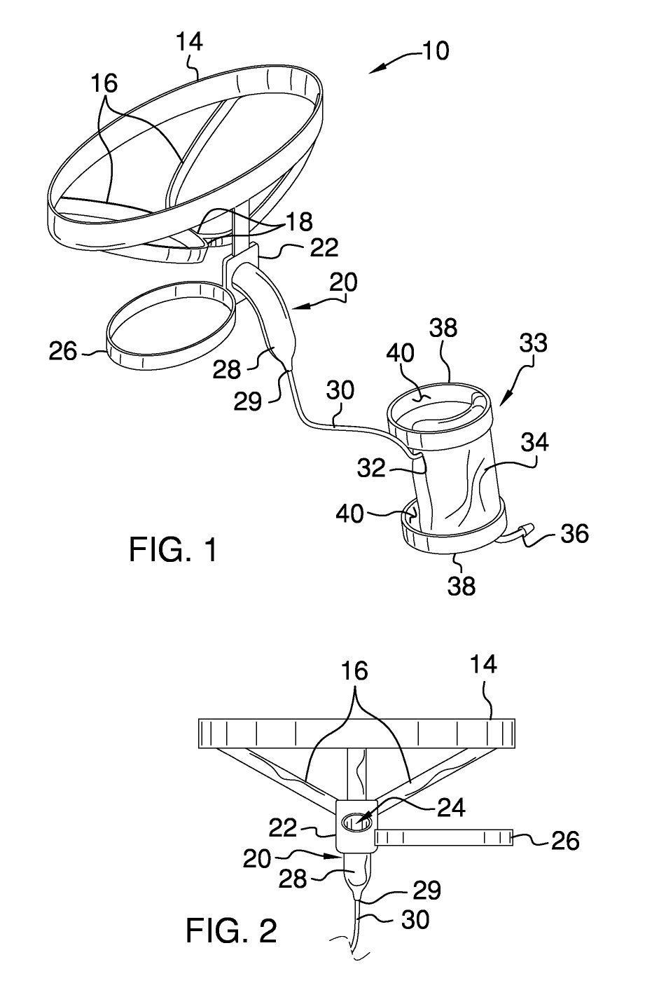

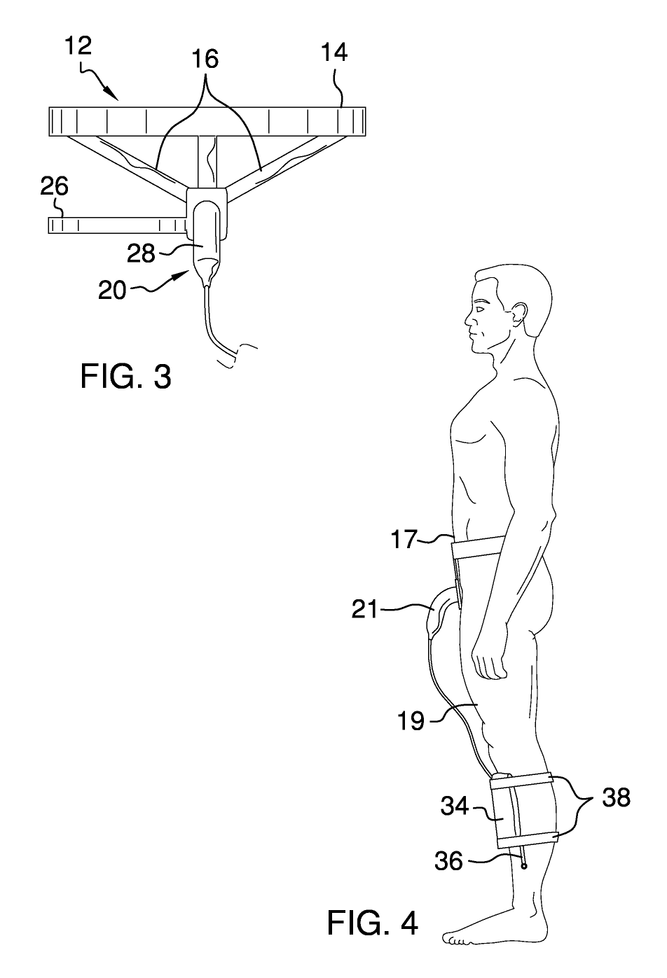

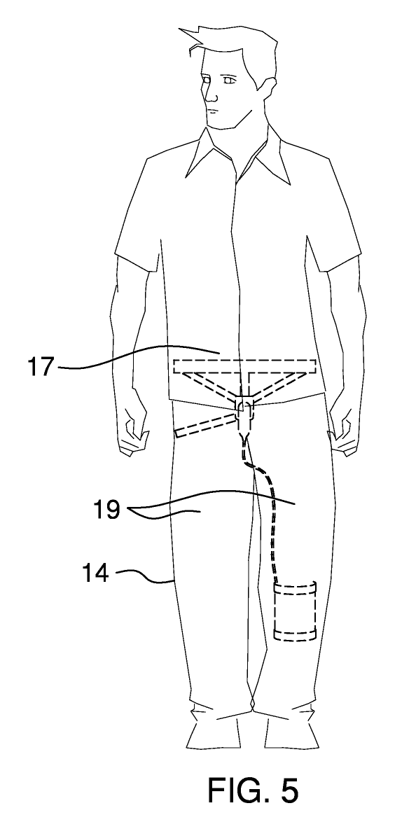

With reference now to the drawings, and in particular to FIGS. 1 through 5 thereof, a new collection device embodying the principles and concepts of an embodiment of the disclosure and generally designated by the reference numeral 10 will be described.

As best illustrated in FIGS. 1 through 5, the urine collection assembly 10 generally comprises a harness 12 that may be worn. The harness 12 has a waist loop 14 and a pair of leg loops 16. Each of the legs loops 16 is coupled to and extends downwardly from the waist loop 14. The leg loops 16 are oppositionally positioned on the waist loop 14. Each of the legs loops 16 has a distal end 18 with respect to the waist loop 14 and the distal end 18 of the legs loops 16 is coupled together. The waist loop 14 may be worn around a waist 17 of a male having each of a pair of legs 19 extending through an associated one of the leg loops 16.

A receptacle 20 is coupled to the harness 12. The receptacle 20 may insertably receive a penis 21 when the harness 12 is worn. Thus, the receptacle 20 may collect urine. The receptacle 20 comprises a panel 22 that is coupled to and extends downwardly from the waist loop 14. The panel 22 is centrally positioned with respect to the leg loops 16. Thus, the panel 22 is aligned with the penis 21 when the harness 12 is worn.

The panel 22 has an aperture 24 extending therethrough. The aperture 24 may have the penis 21 extended therethrough when the harness 12 is worn. A thigh loop 26 is coupled to and extends laterally away from the panel 22. The thigh loop 26 may have one of the legs 19 extending through the thigh loop 26 when the harness 12 is worn.

A sleeve 28 is coupled to the panel 22 and the sleeve 28 is aligned with the aperture 24. Thus, the sleeve 28 may insertably receive the penis 21 when the harness 12 is worn. The sleeve 28 has a distal end 29 with respect to the panel 22. The sleeve 28 may be comprised of a resiliently stretchable and fluid impermeable material such as rubber or the like.

A hose 30 is coupled to the distal end 29 of the sleeve 28. The hose 30 is in fluid communication with the sleeve 28. Thus, the hose 30 directs a flow of urine from the penis 21. The hose 30 has a distal end 32 with respect to the sleeve 28.

A container 33 is provided and the container 33 may be worn. The container 33 is in fluid communication with the receptacle 20 such that the container 33 may store the urine. The container 33 comprises a bag 34 that is fluidly coupled to the distal end 32 of the hose 30. Thus, the bag 34 may contain the urine.

A valve 36 is fluidly coupled to the bag 34 and the valve 36 may be manipulated. The valve 36 is positionable in an open position. Thus, the valve 36 selectively drains the urine from the bag 34. The valve 36 may comprise a manually operated fluid valve or the like.

A pair of hoops 38 is provided and each of the hoops 38 is coupled to the bag 34. Thus, each of the hoops 38 may have one of the legs 19 extended therethrough thereby facilitating the bag 34 to be retained on the leg 19. The hoops 38 are spaced apart from each other and each of the hoops 38 has an inner surface 40. The bag 34 is positioned on the inner surface 40 of each of the hoops 38. Thus, the bag 34 is compressed against the leg when the container 33 is worn thereby facilitating the bag 34 to be substantially concealed with clothing 42.

In use, the harness 12 is worn and the penis 21 is inserted into the sleeve 28. The hoops 38 are positioned around the leg to retain the bag 34 on the leg. The harness 12 and the bag 34 are worn during airline travel or other circumstances that involve limited access to a restroom. The harness 12 and the bag 34 are additionally worn when no restroom facilities are anticipated to be available. The bag 34 is emptied when the bag 34 becomes full of urine.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A urine collection assembly comprising:
a harness being configured to be worn;
a receptacle being coupled to said harness wherein said receptacle is configured to insertably receive a penis when said harness is worn thereby facilitating said receptacle to collect urine; and
a container being configured to be worn, said container being in fluid communication with said receptacle wherein said container is configured to store the urine.

2. The assembly according to claim 1, wherein said harness has a waist loop and a pair of leg loops, each of said legs loops being coupled to and extending downwardly from said waist loop, said leg loops being oppositionally positioned on said waist loop, each of said legs loops having a distal end with respect to said waist loop, said distal end of said legs loops being coupled together, said waist loop being configured to be worn around a waist having each of a pair of legs extending through an associated one of said legs loops.

3. The assembly according to claim 1, wherein:
said harness includes a waist loop and a pair of leg loops; and
said receptacle comprises a panel being coupled to and extending downwardly from said waist loop, said panel being centrally positioned with respect to said leg loops wherein said panel is configured to be aligned with the penis when said harness is worn, said panel having an aperture extending therethrough wherein said aperture is configured to have the penis extended therethrough.

4. The assembly according to claim 3, further comprising a thigh loop being coupled to and extending laterally away from said panel wherein said thigh loop is configured to have one of the legs extending through said thigh loop when said harness is worn.

5. The assembly according to claim 4, further comprising a sleeve being coupled to said panel, said sleeve being aligned with said aperture wherein said sleeve is configured to insertably receive the penis when said harness is worn, said sleeve having a distal end with respect to said panel.

6. The assembly according to claim 5, wherein said sleeve includes a hose being coupled to said distal end of said sleeve, said hose being in fluid communication with said sleeve wherein said hose is configured to direct a flow of urine from the penis, said hose having a distal end with respect to said sleeve.

7. The assembly according to claim 1, wherein:
said receptacle includes a hose, said hose having a distal end; and
said container comprises a bag being fluidly coupled to said distal end of said hose wherein said bag is configured to contain the urine.

8. The assembly according to claim 7, further comprising a pair of hoops, each of said hoops being coupled to said bag wherein each of said hoops is configured to have one of the legs extended therethrough thereby facilitating said bag to be retained on the leg, said hoops being spaced apart from each other.

9. The assembly according to claim 8, wherein each of said hoops has an inner surface, said bag being positioned on said inner surface of each of said hoops wherein said bag is configured to be compressed against the leg when said container is worn thereby facilitating said bag to be substantially concealed with clothing.

10. A urine collection assembly comprising:
a harness being configured to be worn, said harness having a waist loop and a pair of leg loops, each of said legs loops being coupled to and extending downwardly from said waist loop, said leg loops being oppositionally positioned on said waist loop, each of said legs loops having a distal end with respect to said waist loop, said distal end of said legs loops being coupled together, said waist loop being configured to be worn around a waist having each of a pair of legs extending through an associated one of said legs loops; and
a receptacle being coupled to said harness wherein said receptacle is configured to insertably receive a penis when said harness is worn thereby facilitating said receptacle to collect urine, said receptacle comprising:
a panel being coupled to and extending downwardly from said waist loop, said panel being centrally positioned with respect to said leg loops wherein said panel is configured to be aligned with the penis when said harness is worn, said panel having an aperture extending therethrough wherein said aperture is configured to have the penis extended therethrough,
a thigh loop being coupled to and extending laterally away from said panel wherein said thigh loop is configured to have one of the legs extending through said thigh loop when said harness is worn, and
a sleeve being coupled to said panel, said sleeve being aligned with said aperture wherein said sleeve is configured to insertably receive the penis when said harness is worn, said sleeve having a distal end with respect to said panel, said sleeve including a hose being coupled to said distal end of said sleeve, said hose being in fluid communication with said sleeve wherein said hose is configured to direct a flow of urine from the penis, said hose having a distal end with respect to said sleeve; and
a container being configured to be worn, said container being in fluid communication with said receptacle wherein said container is configured to store the urine, said container comprising:
a bag being fluidly coupled to said distal end of said hose wherein said bag is configured to contain the urine,
a valve being fluidly coupled to said bag wherein said valve is configured to be manipulated, said valve being positionable in an open position wherein said valve is configured to selectively drain the urine from said bag, and
a pair of hoops, each of said hoops being coupled to said bag wherein each of said hoops is configured to have one of the legs extended therethrough thereby facilitating said bag to be retained on the leg, said hoops being spaced apart from each other, each of said hoops having an inner surface, said bag being positioned on said inner surface of each of said hoops wherein said bag is configured to be compressed against the leg when said container is worn thereby facilitating said bag to be substantially concealed with clothing.
